(19) United States
(12) Patent Application Publication
(10) Pub. No.: US 2026/0321125 A1
(43) Pub. Date: Oct. 15, 2026

(54) ELECTRONIC DEVICE FOR PROVIDING USER CONDITION INFORMATION RELATED TO VEHICLE ACCIDENT, OPERATING METHOD THEREOF, AND STORAGE MEDIUM

(71) Applicant: Samsung Electronics Co., Ltd., Suwon-si (KR)

(72) Inventors: Jihyeok YOO, Suwon-si (KR); Jinik KIM, Suwon-si (KR); Namjoon PARK, Suwon-si (KR)

(21) Appl. No.: 19/681,840

(22) Filed: May 19, 2026

Related U.S. Application Data
(63) Continuation of application No. PCT/KR2024/016875, filed on Oct. 31, 2024.

(30) Foreign Application Priority Data
Dec. 4, 2023 (KR) 10-2023-0173104
Jan. 22, 2024 (KR) 10-2024-0009437

(51) Int. Cl. G08B 25/01; G06F 21/62; G08B 21/04; G16H 10/65

(52) U.S. Cl. CPC G08B 25/016; G06F 21/6245; G08B 21/0453; G16H 10/65

(57) ABSTRACT

An electronic device is provided. The electronic device includes a display, at least one sensor, at least one processor operatively connected to the display and the at least one sensor, and memory storing instructions that, when executed by the at least one processor individually or collectively, cause the electronic device to, display an indicator for providing status information of a user on the display, based on an occurrence of a vehicle accident being detected while monitoring a driving state of a vehicle using the at least one sensor, and in response to an access to the status information of the user in the vehicle by an external electronic device using the indicator, provide at least a part of the status information of the user.

### CROSS-REFERENCE TO RELATED APPLICATIONS

This application is a continuation application, claiming priority under 35 U.S.C. § 365(c), of an International application No. PCT/KR2024/016875, filed on Oct. 31, 2024, which is based on and claims the benefit of a Korean patent application number 10-2023-0173104, filed on Dec. 4, 2023, in the Ministry of Intellectual Property (MOIP), and of a Korean patent application number 10-2024-0009437, filed on Jan. 22, 2024, in the Ministry of Intellectual Property (MOIP), the disclosure of each of which is incorporated by reference herein in its entirety.

### BACKGROUND

### 1. Field

The disclosure relates to an electronic device for providing vehicle accident-related user status information, an operating method thereof, and a storage medium.

### 2. Description of Related Art

More and more services and additional functions are being provided through electronic devices, e.g., smartphones. For example, the electronic device includes a communication function as well as a sensing function for detecting surrounding circumstances, and may provide more diverse functions through additional installation of an application. An electronic device may not only execute its equipped applications or stored files but also access, wiredly or wirelessly, a server or another electronic device to receive, in real-time, various pieces of information. Accordingly, various functions that are provided through electronic devices are evolving more and more.

Further, a wearable electronic device may include a communication module to independently transmit/receive data, and may be used in conjunction with an electronic device such as a smartphone. The electronic device and/or the wearable electronic device, which are convenient to carry and use, may be exposed to various operating environments. In this case, while a user may more easily use various services using both the electronic device and the wearable electronic device, various privacy and security issues are emerging at the same time, and protection of user information is also becoming important.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide an electronic device for providing vehicle accident-related user status information, an operating method thereof, and a storage medium.

Additional aspects will be set forth in part in the description which follows and, in part, will be apparent from the description, or may be learned by practice of the presented embodiments.

In accordance with an aspect of the disclosure, an electronic device is provided. The electronic device includes a display, at least one sensor, at least one processor operatively connected to the display and the at least one sensor, and memory storing instructions that, when executed by the at least one processor individually or collectively, cause the electronic device to display an indicator for providing status information of a user on the display, based on an occurrence of a vehicle accident being detected while monitoring a driving state of a vehicle using the at least one sensor, and in response to an access to the status information of the user in the vehicle by an external electronic device using the indicator, provide at least a part of the status information of the user.

In accordance with another aspect of the disclosure, a method for providing vehicle accident-related user status information in an electronic device is provided. The method includes displaying an indicator for providing status information of a user in a vehicle, based on an occurrence of a vehicle accident being detected while monitoring a driving state of a vehicle using at least one sensor of the electronic device, and in response to an access to the status information of the user by an external electronic device using the indicator, providing at least a part of the status information of the user.

In accordance with another aspect of the disclosure, one or more non-transitory computer-readable storage media storing one or more computer programs including computer-executable instructions that, when executed by one or more processors of an electronic device individually or collectively, cause the electronic device to perform operations are provided. The operations include displaying an indicator for providing status information of a user in a vehicle, based on an occurrence of a vehicle accident being detected while monitoring a driving state of the vehicle using at least one sensor of the electronic device, and in response to an access to the status information of the user by an external electronic device using the indicator, providing at least a part of the status information of the user.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an electronic device in a network environment according to an embodiment of the disclosure;

FIG. 2A is a system configuration diagram for providing a vehicle accident notification according to an embodiment of the disclosure;

FIG. 2B is a view illustrating an electronic device and a wearable electronic device of a user riding in a vehicle and a wearable electronic device of a passenger according to an embodiment of the disclosure;

FIG. 3 is a view illustrating an internal block configuration diagram illustrating an electronic device for providing vehicle accident-related user status information and a wearable electronic device according to an embodiment of the disclosure;

FIG. 4 is a view illustrating a table classifying vehicle accident-related user status information according to disclosure levels according to an embodiment of the disclosure;

FIG. 5 is an operation flowchart of an electronic device for providing vehicle accident-related user status information according to an embodiment of the disclosure;

FIG. 6 is a detailed operation flowchart of an electronic device for providing vehicle accident-related user status information according to an embodiment of the disclosure;

FIG. 7 is a view illustrating an example of a first screen upon occurrence of a vehicle accident according to an embodiment of the disclosure;

FIG. 8 is a view illustrating an example of a second screen upon occurrence of a vehicle accident according to an embodiment of the disclosure;

FIG. 9A is a view illustrating an example of a screen illustrating vehicle accident information according to an embodiment of the disclosure;

FIG. 9B is a view illustrating a wearable electronic device in a pairing state with an electronic device upon a vehicle accident according to an embodiment of the disclosure;

FIG. 9C is a view illustrating an example of a screen illustrating an indicator for providing vehicle accident-related user status information according to an embodiment of the disclosure;

FIG. 9D is a view illustrating an example of a screen displayed on an external electronic device of an emergency medical technician and/or a medical staff according to an embodiment of the disclosure;

FIG. 10A is an operation flowchart of an electronic device for providing vehicle accident-related user status information with different disclosure levels according to an embodiment of the disclosure;

FIG. 10B is a view illustrating an authentication method according to an embodiment of the disclosure;

FIG. 11 is a view illustrating an example of a screen on which vehicle accident-related user status information with different disclosure levels is provided for each authenticated target using one indicator according to an embodiment of the disclosure;

FIG. 12 is a view illustrating an example of a screen on which health information among user status information is provided according to an embodiment of the disclosure; and

FIG. 13 is a view illustrating an example of a screen on which electronic medical records among user status information are provided according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art to will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a wireless fidelity (Wi-Fi) chip, a Bluetooth^{®} chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

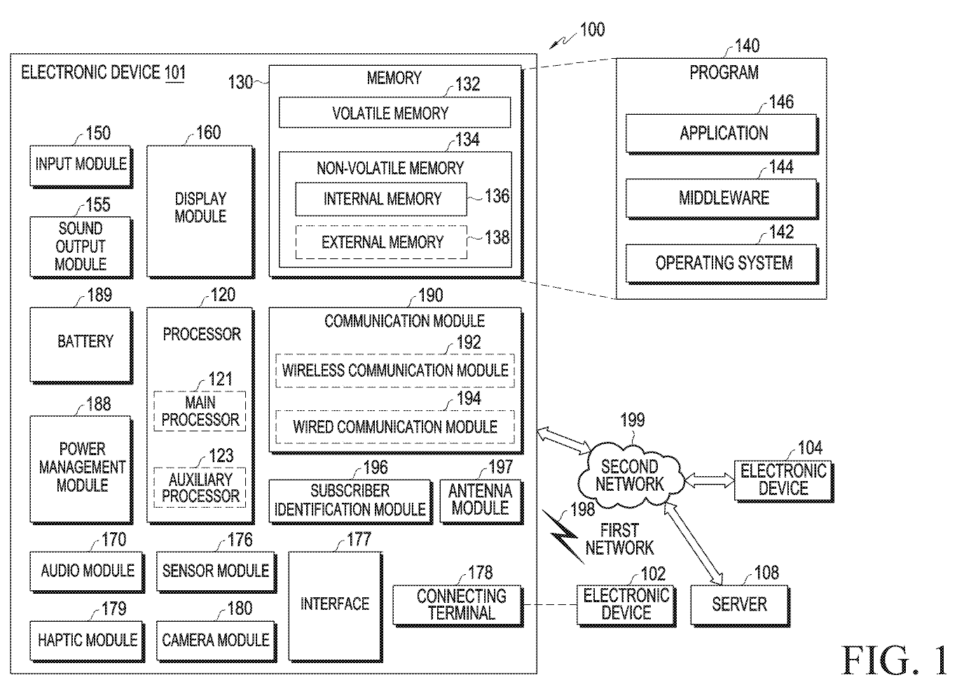

FIG. 1 is a block diagram illustrating an electronic device 101 in a network environment 100 according to an embodiment of the disclosure.

Referring to FIG. 1, the electronic device 101 in the network environment 100 may communicate with at least one of an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In an embodiment, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., the program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by other component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operation state (e.g., power or temperature) of the electronic device 101 or an external environmental state (e.g., the user's state), and then generate an electrical signal or data value corresponding to the detected state. According to an embodiment, the sensor module 176 may include, for example, a gesture sensor, a gyro sensor, an atmospheric pressure sensor, a magnetic sensor, an accelerometer, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wiredly) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wiredly) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth^{TM}, wireless-fidelity (Wi-Fi) direct, or infrared data association (IrDA)) or a second network 199 (e.g., a long-range communication network, such as a legacy cellular network, a fifth generation (5G) network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a fourth generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the millimeter wave (mmWave) band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20Gbps or more) for implementing eMBB, loss coverage (e.g., 164dB or less) for implementing mMTC, or user plane (U-plane) latency (e.g., 0.5ms or less for each of downlink (DL) and uplink (UL), or a round trip of 1ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, instructions or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

In the following description, the components easy to understand from the description of the above embodiments are denoted with or without the same reference numerals and their detailed description may be skipped. According to an embodiment of the disclosure, an electronic device may be implemented by selectively combining configurations of different embodiments, and the configuration of one embodiment may be replaced by the configuration of another embodiment. However, it is noted that the disclosure is not limited to a specific drawing or embodiment.

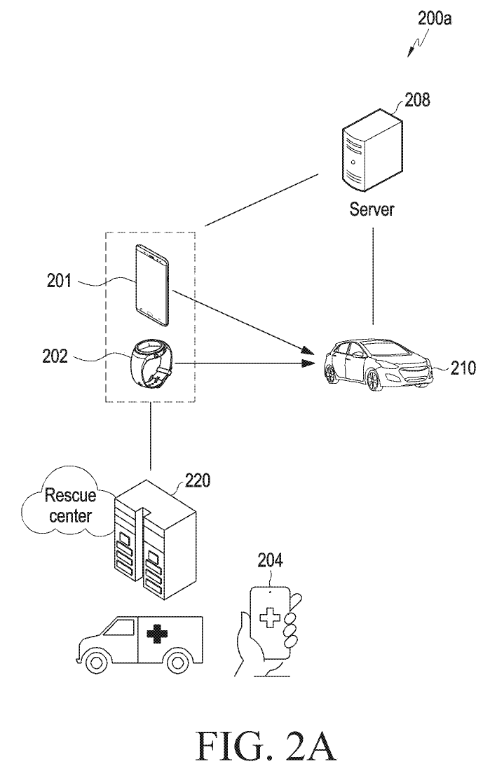

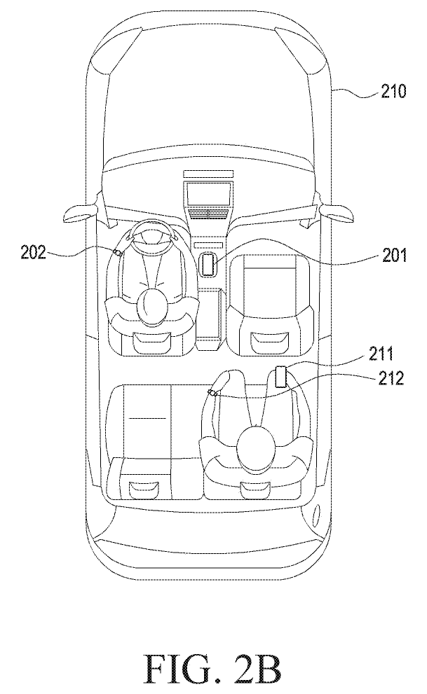

FIG. 2A is a system configuration diagram for providing a vehicle accident notification according to an embodiment of the disclosure.

FIG. 2B is a view illustrating an electronic device and a wearable electronic device of a user riding in a vehicle and a wearable electronic device of a passenger according to an embodiment of the disclosure.

Referring to FIGS. 2A and 2B, a system 200a for providing a vehicle accident notification may include an electronic device 201 (e.g., a smartphone) (e.g., the electronic device 101 of FIG. 1) of a user riding in a vehicle 210. The system 200a may further include a wearable electronic device 202 (e.g., a smart watch) (e.g., the electronic device 102 of FIG. 1) that is linked based on the same user account as the electronic device 201. The system 200a may further include an electronic device 211 and/or a wearable electronic device 212 of a passenger riding in the vehicle 210.

The electronic device 201 may detect an accident circumstance (or collision circumstance) of the vehicle 210 using various sensors included in the electronic device 201. Upon occurrence of a vehicle accident, the electronic device 201 may notify an emergency circumstance, and may determine an actual accident circumstance according to user feedback. Accordingly, the electronic device 201 supporting an emergency circumstance notification may request emergency rescue to a rescue center (or emergency dispatch agency) 220, or may deliver the emergency circumstance by communicating with a server 208 through a communication network.

As such, in order to minimize casualties due to a vehicle accident, it may be necessary to quickly report to the rescue center 220 and promptly handle and manage the accident. For this purpose, in an emergency circumstance where an emergency patient such as a driver or a passenger due to a vehicle accident loses consciousness, information such as a medical history or an underlying disease of the emergency patient may not be known, and thus treatment may be performed without considering risk factors during emergency treatment. In preparation for such an emergency circumstance, when a user has pre-registered medical information as well as personal information in an electronic device, the electronic device may provide a function that allows viewing of the registered information by an external electronic device 204 of an external user.

However, since sensitive medical information may be exposed to an external user regardless of user intent, security of sensitive information such as personal information and medical information of an emergency patient may be very important in an emergency circumstance.

Therefore, upon occurrence of a vehicle accident, there may be a need for a method capable of minimizing exposure of user status information related to vehicle accident while enabling the electronic device to quickly perform various responses based on various accident information such as an accident type or passenger information.

An embodiment relates to an electronic device for effectively providing vehicle accident-related user status information so as to minimize exposure of information about a user of the electronic device to an unauthorized external user upon occurrence of a vehicle accident, an operating method thereof, and a storage medium.

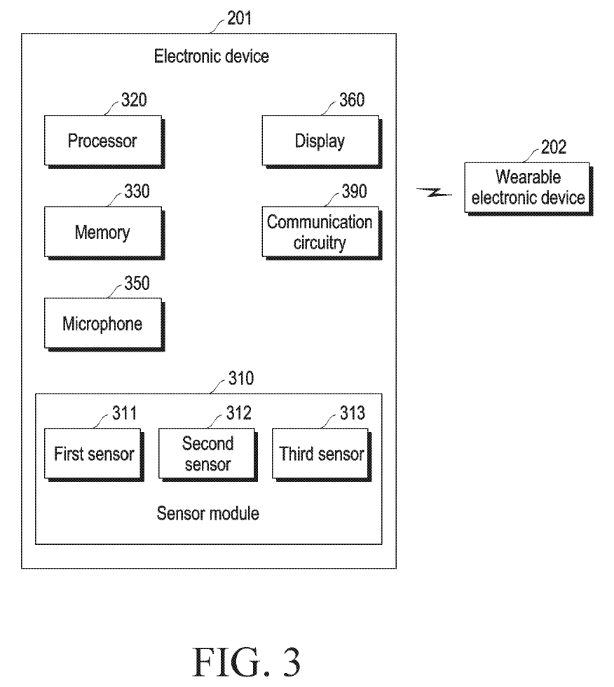

FIG. 3 is a view illustrating an internal block configuration diagram illustrating an electronic device for providing vehicle accident-related user status information and a wearable electronic device according to an embodiment of the disclosure.

Referring to FIG. 3, an electronic device 201 (e.g., the electronic device 101 of FIG. 1 or the electronic device 201 of FIGS. 2A and 2B) may include a sensor module 310 (e.g., the sensor module 176 of FIG. 1), at least one processor 320 (e.g., the processor 120 of FIG. 1), memory 330 (e.g., the memory 130 of FIG. 1), and a display 360 (e.g., the display module 160 of FIG. 1). The sensor module 310 may include at least one sensor 311, 312, 313. According to an embodiment, the electronic device 201 may further include at least one microphone 350 (e.g., the input module 150 of FIG. 1) or communication circuitry 390 (e.g., the communication module 190 of FIG. 1). According to an embodiment, the electronic device 201 may omit at least one of the components or may additionally include other components.

According to an embodiment, the sensor module 310 may sense information (or data) for identifying a collision and/or an accident of a vehicle (e.g., the vehicle 210 of FIGS. 2A and 2B). The sensor module 310 may include a first sensor 311, a second sensor 312, and/or a third sensor 313. For example, the first sensor 311 may include an acceleration sensor for sensing acceleration. The second sensor 312 may include a sound sensor for sensing a vehicle collision sound. The third sensor 313 may include a barometric pressure sensor for sensing barometric pressure. Meanwhile, the sensors 311, 312, 313 illustrated in FIG. 3 are merely, and the sensors included in the sensor module 310 may not be limited thereto.

According to an embodiment, the processor 320 may control the overall operation of the electronic device 201. For example, the processor 320 may be implemented identically or similarly to the processor 120 of FIG. 1.

According to an embodiment, the processor 320 may establish a communication connection to a wearable electronic device 202 worn on a user's body through the communication circuitry 390. For example, the communication circuitry 390 may support short-range communication technology (e.g., Bluetooth, Bluetooth low energy (BLE), Wi-Fi). Further, the communication circuitry 390 may support ultra wide band (UWB) communication technology. The communication connection may be formed using any one of the communication technologies supported by the communication circuitry 390. For example, the wearable electronic device 202 may be implemented as a smart watch that may be worn on a user's wrist. Further, the wearable electronic device 202 may include a device configured to be wearable on a user's body in addition to a smart watch, such as a smart ring, a head-mounted display, or glasses-type augmented reality (AR) glasses device, and is not limited thereto, and may be implemented in various forms that may be worn on a user's body.

According to an embodiment, the processor 320 may identify that a user is riding in a vehicle 210 through at least one of the sensor module 310 or the communication circuitry 390.

In an embodiment, the processor 320 may identify whether a user is riding in the vehicle 210 based on barometric pressure change data according to door opening or door closing of the vehicle 210 through the sensor module 310 (e.g., a barometric pressure sensor).

In an embodiment, the processor 320 may identify whether a user is riding in the vehicle 210 based on global navigation satellite system (GNSS) information or cell switching information. For this purpose, the processor 320 may detect movement at a predetermined speed or higher or check cell switching information through a GNSS module (not illustrated) or a GPS module (not illustrated). Here, the GNSS module may receive a signal capable of identifying a location of the electronic device 201. For example, the GNSS module may receive a signal capable of identifying a location of the electronic device 201 using at least one of GPS, GLONASS, Galileo, or Beidou.

In an embodiment, in addition to the above, the processor 320 may detect a vibration pattern of the vehicle 210 using the sensor module 310 (e.g., an acceleration sensor or a gyro sensor), detect a magnetic pattern, or detect a pattern of engine sound of the vehicle 210 and/or a low-frequency sound output from the vehicle 210 using a sound sensor. The processor 320 may identify or determine whether a user is riding in the vehicle 210 based on the detected pattern and/or information.

Further, when connected to a settop device (not illustrated) mounted in the vehicle 210 (e.g., a vehicle application installed in the settop device) or when connected to the settop device using Bluetooth communication, the processor 320 may determine that a user is riding in the vehicle 210. For example, when approaching within a predetermined distance (e.g., a communication-possible range) from the vehicle 210, the processor 320 may be connected to the vehicle 210 through various communication methods (e.g., Bluetooth, low power Bluetooth, or UWB). The processor 320 may be connected to the vehicle 210 through Bluetooth communication when power is turned on or an engine is started in the vehicle 210. When attempting to connect to the vehicle 210 through a scanning process (e.g., BLE advertising operation) or when connected to the vehicle 210 through a configured communication method (e.g., Bluetooth or UWB), the processor 320 may determine that a user is riding in the vehicle 210. Alternatively, when location information of the vehicle 210 upon getting off the vehicle 210 is stored in the memory 330, the processor 320 may determine that a user is approaching or riding in the vehicle 210 when location information of the electronic device 201 obtained from a GNSS module is within a predetermined distance (e.g., about 1 m, about 30 cm) from the location information of the vehicle 210.

As described above, a method of identifying whether a user is riding in the vehicle 210 may not be limited to the foregoing. Further, an operation of identifying whether a user is riding in the vehicle 210 may be performed substantially identically in the wearable electronic device 202 as well as the electronic device 201. For example, in a case where the wearable electronic device 202 includes communication circuitry and may independently transmit/receive data, when connected to a settop device (not illustrated) mounted in the vehicle 210 (e.g., a vehicle application installed in the settop device) or when connected to the settop device using Bluetooth communication, the wearable electronic device 202 may determine that a user wearing the wearable electronic device 202 is riding in the vehicle 210. Further, when the electronic device 201 is in a state of being communication-connected with the wearable electronic device 202, the electronic device 201 may receive information sensed by the wearable electronic device 202 from the wearable electronic device 202, and may identify whether a user is riding in the vehicle 210 based on the received information. As described above, an identification operation of whether a user is riding in the vehicle 210 may be performed by the electronic device 201 and/or the wearable electronic device 202.

According to an embodiment, in a state of identifying that a user is riding in the vehicle 210, the processor 320 may identify information about a user riding in the vehicle 210. Since at least one user may ride in the vehicle 210, if information about a passenger as well as a user may be identified, more efficient response may be possible when requesting emergency rescue.

In an embodiment, in a state of detecting that a user of the electronic device 101 is riding in the vehicle 210, the processor 320 may detect a direction of sound or external sound through at least one microphone 350. Accordingly, when a user speaks, the processor 320 may identify the number of persons riding in the vehicle 210 based on a voice signal received through the microphone 350. Meanwhile, the processor 320 may identify a user riding in the vehicle 210 through various methods other than a method using a voice signal.

In an embodiment, in a state of identifying that a user is in the vehicle 210, the processor 320 may monitor a driving state of the vehicle 210. For example, the processor 320 may identify that a user is riding in the vehicle 210 according to being connected to the vehicle 210 through Bluetooth communication, and may monitor a driving state using speed based on GPS when the vehicle 210 moves.

In an embodiment, the processor 320 may simultaneously monitor whether a user is riding in the vehicle 210 and a driving state of the vehicle 210. For example, the processor 320 may identify that a user is riding in the vehicle 210 using at least one sensor of the sensor module 310 and/or GPS, and may monitor a driving state using speed based on GPS. As such, an operation of monitoring whether a user is riding in the vehicle 210 and a driving state may be performed independently or may be performed as one operation.

As described above, the processor 320 may recognize whether a user is riding in the vehicle 210 and a driving state of the vehicle 210 using various methods (e.g., communication connection, movement pattern, or speed using GPS). The processor 320 may detect an occurrence of a vehicle accident while monitoring a driving state of the vehicle 210. In an embodiment, the processor 320 may periodically or aperiodically monitor a driving state of the vehicle 210 using at least one sensor of the sensor module 310 and/or GPS. Here, the driving state of the vehicle 210 may include at least one of a traveling direction of the vehicle 210, a speed of the vehicle 210, a direction change of the vehicle 210, an acceleration direction of the vehicle 210, or a rotation direction of the vehicle 210.

In an embodiment, when an acceleration value sensed through the sensor module 310 (e.g., an acceleration sensor, a gyro sensor) is equal to or greater than a predetermined threshold while monitoring a driving state of the vehicle 210, a GNSS movement speed sharply decreases, and a sound suspected of being a collision of the vehicle 210 is detected, the processor 320 may identify that the vehicle 210 has collided or an accident occurs. The processor 320 may identify whether a barometric pressure change amount is equal to or greater than a predetermined threshold through the sensor module 310 (e.g., a barometric pressure sensor). For example, when a window is broken or an airbag is deployed due to a collision of the vehicle 210, a barometric pressure of the vehicle 210 may change sharply, so the processor 320 may detect a barometric pressure change. The processor 320 may detect whether the vehicle 210 has collided (or whether an accident occurs) based on the above-described sensing values.

According to an embodiment, the processor 320 may detect whether the vehicle 210 has collided through such monitoring. As such, although at least one sensor of the sensor module 310 and/or GPS each senses (or obtains) information related to a state of the vehicle 210, a component (or module) for obtaining a vehicle state and a component (or module) for detecting a vehicle collision may be operated in parallel. For example, even with the same sensing information (e.g., sensing information by the sensor module 310, GPS information), analysis and processing may be performed differently for the purpose of determining a vehicle state and for the purpose of detecting a vehicle collision.

According to an embodiment, the memory 330 may store instructions for providing status information of a user upon occurrence of a vehicle accident, and may be the memory 130 described above with reference to FIG. 1. In an embodiment, the processor 320 may store various information about a user in the memory 330. For example, the processor 320 may pre-store various information necessary for emergency treatment of a user as well as personal information of a user upon a vehicle accident. Various information about a user may be referred to as status information of a user or emergency information about a user.

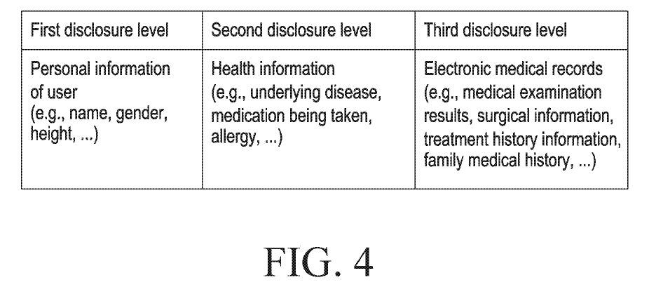

FIG. 4 may be referred to illustrating the status information of a user.

FIG. 4 is a view illustrating a table classifying vehicle accident-related user status information according to disclosure levels according to an embodiment of the disclosure.

Referring to FIG. 4, in order to prevent status information of a user from being misused by an unauthorized external user and to prevent privacy infringement, the status information of a user may be largely classified into three levels according to disclosure levels. The three disclosure levels in FIG. 4 are merely, and the disclosure levels may be divided into two levels or four levels, so a step of classifying disclosure levels may not be limited thereto.

FIG. 4 illustrates a case where status information of a user is classified into three levels according to disclosure levels, and the status information of a user may include personal information directly input by a user, health information about a user necessary for emergency treatment in preparation for an emergency circumstance, and electronic medical records indicating medical information about a user.

For example, personal information corresponding to a first disclosure level may include personal information necessary for identifying a user's identity, such as a user's name, gender, and height. Health information corresponding to a second disclosure level may include emergency information necessary for emergency treatment, such as an underlying disease, a medication being taken, and an allergy of a user. Electronic medical records (EMR) corresponding to a third disclosure level may include all medical information such as medical examination results, surgical information, treatment history information, and family medical history. The electronic medical records may be designed to be used by a specific organization or medical staff having access authority. Medical information corresponding to a third disclosure level may be electronic health records (EHR) in addition to electronic medical records or may include a combination of electronic medical records and electronic health records.

In an embodiment, among status information of a user, personal information of a user at a first disclosure level and health information of a user at a second disclosure level may be information directly recorded by a user according to input items provided by the electronic device 201. On the other hand, among status information of a user, electronic medical records at a third disclosure level may be information provided from a separate server (e.g., a medical institution) that manages medical information.

The status information of a user may be stored in the memory 330 and/or a server 208 accessible by an authenticated external electronic device. When status information of a user is stored in the memory 330 in the electronic device 201, security is high, and the status information of a user may be provided even in a circumstance where a network connection is not possible. On the other hand, when status information of a user is stored in the server 208, information necessary for emergency treatment of a user who is an emergency patient may be easily shared between an emergency medical technician and a medical staff. According to an embodiment, security of medical information and efficiency for prompt emergency treatment may be simultaneously provided through a method of storing in each of the electronic device 201 and the server 208.

In an embodiment, the status information of a user may be encrypted and stored after being classified into personal information, health information, and electronic medical records to correspond to each of the first disclosure level to the third disclosure level as illustrated in FIG. 4.

In an embodiment, the status information of a user may be stored in an encrypted form and may be classified according to an access authority of an external electronic device upon access by the external electronic device in the event of a future vehicle accident. In an embodiment, when using a learning model, learning may be performed to have determination criteria on how to classify using information included in status information of a user. For example, the learning model may infer, based on vehicle accident information and status information of a user, whether an emergency circumstance of a user is a circumstance requiring emergency treatment by an emergency medical technician or a circumstance requiring diagnosis and treatment by hospital medical staff. Accordingly, the learning model may check information necessary in an inferred circumstance among status information of a user and may classify the checked information according to disclosure levels.

The processor 320 may control processing of input data according to a predefined operation rule or a learning model stored in the memory 330. The predefined operation rule or the learning model is characterized by being created through learning. Here, "created by training" means that a predefined operation rule or artificial intelligence model configured to achieve a desired feature (or goal) is created by training a default artificial intelligence model with multiple pieces of training data and a training algorithm. Such learning may be performed in the electronic device 201 according to the disclosure itself, or may be performed through a separate server, e.g., the server 208 accessible by an authenticated external electronic device and/or a system. Examples of the learning algorithm include supervised learning, unsupervised learning, semi-supervised learning, reinforcement learning, or machine learning, but are not limited to the foregoing examples.

Through the above-described method, the processor 320 may output information classified according to disclosure levels not directly on the display 360, but on the display 360 after authentication according to an access authority of an external electronic device upon access by the external electronic device after occurrence of a vehicle accident.

Meanwhile, the processor 320 may periodically or aperiodically monitor a driving state of the vehicle 210 using at least one sensor of the sensor module 310 in order to identify whether a vehicle accident occurs. For example, the processor 320 may periodically or aperiodically monitor a traveling direction and speed of the vehicle 210. Here, the driving state of the vehicle 210 may include at least one of a traveling direction of the vehicle 210, a speed of the vehicle 210, a direction change of the vehicle 210, an acceleration direction of the vehicle 210, or a rotation direction of the vehicle 210.

According to an embodiment, in a state in which a user is riding in the vehicle 210 (e.g., a state in which the vehicle 210 is driving), when an acceleration value sensed through the sensor module 310 (e.g., an acceleration sensor, a gyro sensor) is equal to or greater than a predetermined threshold, a GNSS movement speed sharply decreases, and a sound suspected of being a collision of the vehicle 210 is detected, the processor 320 may identify that the vehicle 210 has collided or an accident occurs. For example, the processor 320 may capture audio data for a designated time (e.g., about 7 seconds) before and after an accident circumstance and perform frequency analysis. For example, using an audio digital signal processor (ADSP), a voice signal of a user may be constantly received through the microphone 350 in a low-power process. If noise exceeding a threshold occurs, the processor 320 may identify that the vehicle 210 has collided or an accident occurs by searching for characteristic noise (e.g., human screaming, window breaking sound) generated when an accident occurs through machine learning based on a neural network. However, GNSS information may be used to prevent false recognition in a circumstance similar to a vehicle accident circumstance. For example, when a GNSS movement speed sharply decreases and then increases again, it may be classified as not an accident circumstance and, when GNSS information indicates a place where vehicle operation is not possible (e.g., inside an amusement park or inside a ski resort other than a parking lot), it may also be classified as not an accident circumstance.

Further, the processor 320 may identify whether an angular velocity change amount is equal to or greater than a predetermined threshold through the sensor module 310. For example, when a traveling direction of the vehicle 210 is sharply twisted in a different direction due to a collision, the processor 320 may detect a sharp angular velocity change through the sensor module 310. Further, since a pattern having a significant meaning in horizontal movement is generated due to a collision of the vehicle 210, the processor 320 may detect an angular velocity change by adjusting or increasing a weight for horizontal movement (or impulse) of a navigation frame sensed by the sensor module 310.

The processor 320 may detect whether the vehicle 210 has had an accident (or whether a collision occurs) based on the above-described sensing values. Further, the processor 320 may identify at least one of an estimated accident time, an occurrence location, an accident type classified as front collision, rear collision, side collision, or rollover of the vehicle 210, the number of persons riding in the vehicle 210, driver and/or passenger information, or accident intensity based on the above-described sensing values and GPS information.

In an embodiment, the processor 320 may estimate accident intensity based on sensing values (or sensing data) sensed by the sensor module 310 using an AI probability model (or learning model). For example, data such as an inertial sensor impact amount at the time of collision, the number of passengers, and/or a magnitude of audio data may be obtained as learning data. Further, injury risk according to accident type and a barometric pressure sensor change amount may also be used as learning data. Since injury probability differs according to accident type, an accident of a type with a high injury probability may have high accident intensity. For example, a large change in a barometric pressure sensor is related to glass breakage, so the data may also be used as learning data as an indicator that may indicate severity of a traffic accident. The AI probability model may calculate a weight of each characteristic through regression analysis and may classify accident intensity as high, medium, and low based on a result score. For example, a high-intensity accident may indicate that a probability of serious injury or higher is high, a medium-intensity accident may indicate that various injuries from minor injury to serious injury may occur, and a low-intensity accident may mean that a possibility of injury due to an accident is low.

According to an embodiment, based on detecting the occurrence of the vehicle 210 accident, the processor 320 may display vehicle accident information through the display 360. Alternatively, the processor 320 may provide a notification indicating an accident to a user through a vibration element, and a method of outputting a notification is not limited thereto. For example, the processor 320 may provide a notification indicating an accident through an audio notification such as a warning sound or a notification outputting accident content by voice.

In an embodiment, the processor 320 may provide a notification based on vehicle accident information and identified status information of a user in the vehicle 210. In a state of being connected with the wearable electronic device 202, the processor 320 may provide the notification to the wearable electronic device 202 so that the notification is displayed through a display of the wearable electronic device 202.

For example, the notification may include at least one of an estimated accident time, an occurrence location, an accident type classified as front collision, rear collision, side collision, or rollover of the vehicle 210, the number of persons riding in the vehicle 210, driver and/or passenger information, accident intensity, biosignal information of a user, or status information of a user. Further, the notification may include an item for selecting whether to perform emergency rescue, a designated institution, or a designated contact (e.g., family, 119).

The processor 320 may selectively provide a rescue notification according to user feedback on the notification. For example, in the case of a minor accident, a user may select cancellation of an emergency contact in response to the notification. On the other hand, if it is identified that a user is in a high-risk state where emergency rescue may not be requested, an emergency call may be performed to a designated institution or a designated contact (e.g., family, 119).

In an embodiment, considering a case where a user has lost consciousness due to a collision of the vehicle 210, the processor 320 may operate so that emergency rescue is automatically performed based on biosignal information of a user.

In an embodiment, when a collision of the vehicle 210 is detected, the processor 320 may transmit a signal for obtaining information (e.g., sensing information) corresponding to a time where the collision occurred to the wearable electronic device 202. The processor 320 may obtain or check information sensed about a user from the wearable electronic device 202, such as biosignal information such as blood pressure, blood oxygen saturation (SpO2), and heart rate. Accordingly, the processor 320 may identify an abnormal condition of a user wearing the wearable electronic device 202 based on the biosignal information.

If the abnormal condition of a user is an emergency state, the processor 320 may perform an emergency call. The processor 320 may transmit an emergency contact and/or a rescue signal to a pre-designated institution or a designated contact (e.g., family, 119). Further, the processor 320 may transmit accident information indicating an accident circumstance due to a collision of the vehicle 210 to the server 208. For example, the server 208 may be a server managed by a designated service provider, a server managed by an insurance company, or a server managed by an automobile manufacturer.

Accordingly, in an embodiment, more accurate guidance about a vehicle collision and/or an accident circumstance may be possible using various information such as a vehicle 210 accident occurrence point, a collision location with respect to the vehicle 210, the number of persons riding in the vehicle 210, driver and/or passenger information, or biosignal information of a user.

In an embodiment, when providing the notification, the processor 320 may provide only at least a part of the status information of the user for privacy protection. The processor 320 may display at least a part of status information of a user on the display 360 in response to an access (or disclosure request) to the status information of the user from an electronic device of an authenticated external user such as an emergency medical technician or a medical staff, i.e., an external electronic device (e.g., the external electronic device 204 of FIG. 2A). The processor 320 may authenticate the external electronic device in response to the access to the status information of the user. In other words, the processor 320 may identify whether the external electronic device is a device of an authenticated target capable of viewing the status information of the user. The processor 320 may identify a disclosure level of status information of a user in relation to an authenticated target in response to the access. The processor 320 may identify an access authority of an external electronic device and may check a disclosure level corresponding to the identified access authority.

As such, authenticating an external electronic device in response to an access to status information of a user is for preventing exposure of sensitive information and enhancing security and privacy protection by allowing access to a part or all of status information of a user at a permitted disclosure level for a target having an access authority.

The processor 320 may display status information of a user on the display 360 using an indicator to provide at least a part of the status information of a user based on a disclosure level. For example, the indicator may be a path accessible to status information of a user. Further, the indicator may include a unique encrypted quick response (QR) code, an address code, a uniform resource locator (URL), a barcode, or a secret string to be used to access status information of a user.

The processor 320 may determine a disclosure level of status information of a user according to an access authority of an external electronic device, but may also determine the disclosure level according to various circumstances. For example, after emergency transport is started and before arriving at a medical institution (e.g., a hospital), an emergency medical technician may be provided with information necessary for emergency treatment among status information of a user, and after a time of arrival at a medical institution, a medical staff may need to be provided with information necessary for diagnosis and treatment among status information of a user. Accordingly, the processor 320 may determine a time of emergency transport or a time of arrival at a medical institution using sensing information by the sensor module 310 and location information (e.g., GPS information) of an electronic device.

Based on identifying a time of emergency transport, the processor 320 may provide health information of a user necessary for emergency treatment in response to an access by an external electronic device of an emergency medical technician. Further, based on identifying a time of arrival at a medical institution, the processor 320 may provide electronic medical records of a user necessary for diagnosis and treatment in response to an access by an external electronic device of a medical staff.

Meanwhile, upon occurrence of a vehicle accident, the electronic device 201 may be disposed at a location separated from a user due to various factors. In such a case, in order for an emergency medical technician to identify whether the electronic device 201 belongs to a user when examining a state of a user, common information indicating that the electronic device 201 and the wearable electronic device 202 are in a paired state may be displayed on each of the electronic device 201 and the wearable electronic device 202. For example, information commonly displayed on each of the electronic device 201 and the wearable electronic device 202 may include a specific code, and any information capable of indicating that they are devices of the same user may be possible. For example, if a user's name is simultaneously displayed on each of the electronic device 201 and the wearable electronic device 202, an external user may recognize that they are devices of the same user, so a form of the commonly displayable information may not be limited thereto.

In an embodiment, by providing information necessary for emergency treatment and information necessary for precise diagnosis and treatment separately, an emergency medical technician may reduce time required for grasping accident circumstances and patient conditions, and even after hospital transfer, omission of patient medical information may be minimized, so efficient response may be possible at a vehicle accident scene.

In an embodiment, a display output function for outputting accident information and status information of a user may be activated only when a vehicle accident occurs. Further, only an authorized external user such as an emergency medical technician or a medical staff may access status information of a user through an encrypted QR code, exposure to an unauthorized external user may be minimized, and a privacy problem may be solved.

According to an embodiment, an electronic device 101, 201 may include a display 160, 360, at least one sensor 176, 310, at least one processor 120, 320 operatively connected to the display and the at least one sensor, and memory 130, 330. According to an embodiment, the memory may be configured to, when executed by the at least one processor, cause the electronic device to display an indicator for providing status information of a user in a vehicle on the display, based on an occurrence of a vehicle accident being detected while monitoring a driving state of a vehicle 210 using the at least one sensor.

According to an embodiment, the instructions may be configured to cause the electronic device to provide at least a part of the status information of the user in response to an access by an external electronic device to the status information of the user using the indicator.

According to an embodiment, the instructions may be configured to cause the electronic device to identify a disclosure level of the status information of the user in response to an access by an external electronic device to the status information of the user using the indicator, and display at least a part of the status information of the user on the display based on the identified disclosure level.

According to an embodiment, the status information of a user may include personal information of a user, health information recorded by a user, or electronic medical records of a user.

According to an embodiment, the status information of a user may be stored in the memory or a server accessible by an external electronic device.

According to an embodiment, the instructions may be configured to cause the electronic device to identify a disclosure level of the status information of the user according to an access authority of an external electronic device, display the personal information of the user among the status information of the user on the display based on the identified disclosure level being a first disclosure level, display the health information recorded by the user among the status information of the user on the display based on the identified disclosure level being a second disclosure level, and display the electronic medical records of the user among the status information of the user on the display based on the identified disclosure level being a third disclosure level.

According to an embodiment, the instructions may be configured to cause the electronic device to identify a time of emergency transport or a time of arrival at a medical institution based on sensing information by the at least one sensor and location information of the electronic device in response to an access by an external electronic device to the status information of the user using the indicator, display the health information recorded by the user among the status information of the user on the display based on identifying the time of emergency transport, and display the electronic medical records of the user among the status information of the user on the display based on identifying the time of arrival at the medical institution.

According to an embodiment, the indicator for providing status information of a user may include a unique encrypted QR code to be used to access the status information of the user.

According to an embodiment, the electronic device may further include communication circuitry 190, 390, and the instructions may be configured to cause the electronic device to, in case that the electronic device 201 is connected to a wearable electronic device 202 worn by a user through the communication circuitry, identify an abnormal condition of a user based on a biosignal of a user provided from the wearable electronic device based on detecting occurrence of a vehicle accident, and perform an emergency call based on the abnormal condition of a user.

According to an embodiment, the instructions may be configured to cause the electronic device to, in case that the electronic device 201 is connected to a wearable electronic device 202 worn by a user through the communication circuitry, display a specific code common to the wearable electronic device 202 on the display to indicate a pairing state between the electronic device and the wearable electronic device.

According to an embodiment, the instructions may be configured to cause the electronic device to identify whether a user is in a vehicle 210 using at least one of the at least one sensor or the communication circuitry, and monitor a driving state of the vehicle based on identifying that a user is in the vehicle.

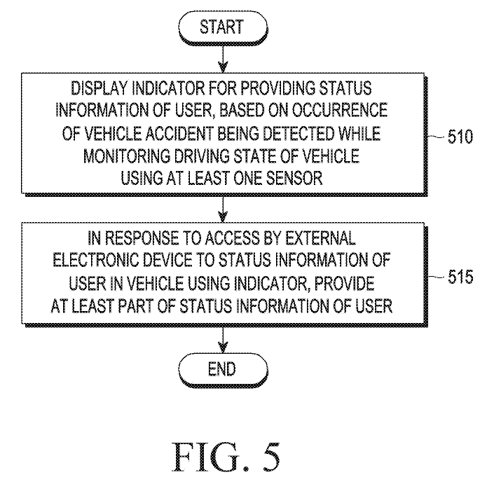

FIG. 5 is an operation flowchart of an electronic device for providing vehicle accident-related user status information according to an embodiment of the disclosure.

Referring to FIG. 5, an operation method may include operations 505 to 515. Each operation of the operation method of FIG. 5 may be performed by at least one of an electronic device (e.g., the electronic device 101 of FIG. 1, the electronic device 201 of FIGS. 2A, 2B, and 3) and at least one processor of the electronic device (e.g., the processor 120 of FIG. 1 or the processor 320 of FIG. 3). In an embodiment, at least one of operations 505 or 515 may be omitted, an order of some operations may be changed, another operation may be added, or at least two operations may be performed in parallel.

Referring to FIG. 5, in operation 510, the electronic device 201 may display an indicator for providing status information of a user in a vehicle, based on an occurrence of a vehicle accident being detected while monitoring a driving state of a vehicle 210 using at least one sensor of the electronic device 201.

According to an embodiment, the indicator for providing status information of a user may include a unique encrypted QR code to be used to access the status information of the user.

According to an embodiment, the status information of a user may include personal information of a user, health information recorded by a user, or electronic medical records of a user.

According to an embodiment, the status information of a user may be stored in the memory or a server accessible by an external electronic device.

According to an embodiment, in case that the electronic device 201 is connected to a wearable electronic device 202 worn by a user, the electronic device 201 may identify an abnormal condition of a user based on a biosignal of the wearable electronic device based on detecting occurrence of a vehicle accident. The electronic device 201 may perform an emergency call based on the abnormal condition of a user.

According to an embodiment, in case that the electronic device 201 is connected to a wearable electronic device 202 worn by a user, the electronic device 201 may display a specific code common to the wearable electronic device 202 to indicate a pairing state between the electronic device and the wearable electronic device.

In operation 515, the electronic device 201 may provide at least a part of the status information of a user in response to an access by an external electronic device (e.g., the external electronic device 204 of FIG. 2A) to the status information of a user using the indicator.

According to an embodiment, in response to an access by an external electronic device to the status information of a user using the indicator, the electronic device 201 may identify a disclosure level of the status information of a user. According to an embodiment, the electronic device 201 may display at least a part of status information of a user based on the identified disclosure level.

According to an embodiment, the electronic device 201 may identify a disclosure level of status information of a user according to an access authority of an external electronic device.

According to an embodiment, based on the identified disclosure level being a first disclosure level, the electronic device 201 may display personal information of a user among status information of a user.

According to an embodiment, based on the identified disclosure level being a second disclosure level, the electronic device 201 may display health information recorded by a user among status information of a user.

According to an embodiment, based on the identified disclosure level being a third disclosure level, the electronic device 201 may display electronic medical records of a user among status information of a user.

According to an embodiment, in response to an access by an external electronic device to status information of a user using the indicator, the electronic device 201 may identify a time of emergency transport or a time of arrival at a medical institution based on sensing information by the at least one sensor and location information of the electronic device. According to an embodiment, based on identifying a time of emergency transport, the electronic device 201 may display health information recorded by a user among status information of a user. According to an embodiment, based on identifying a time of arrival at a medical institution, the electronic device 201 may display electronic medical records of a user among status information of a user.

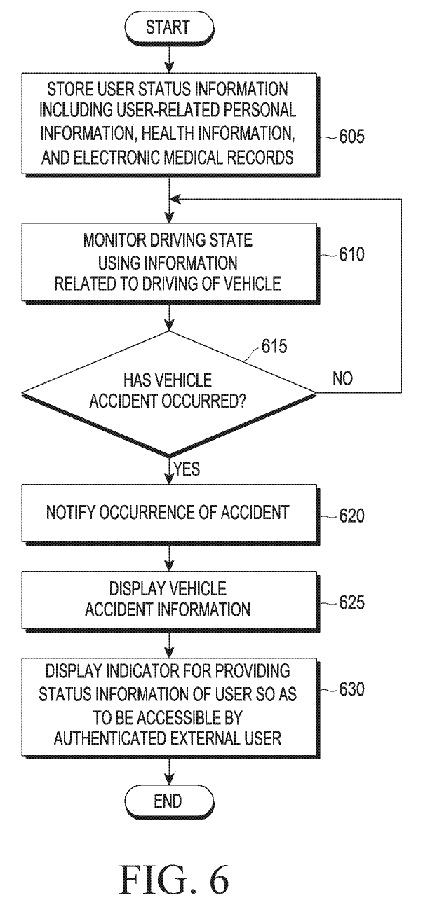

FIG. 6 is a detailed operation flowchart of an electronic device for providing vehicle accident-related user status information according to an embodiment of the disclosure.

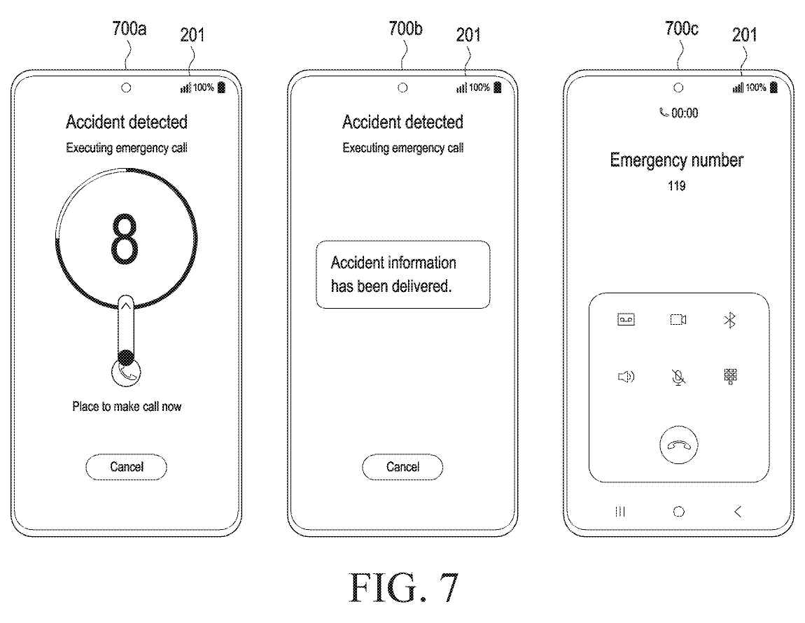

Referring to FIG. 6, an operation method may include operations 605 to 630. Each operation of the operation method of FIG. 6 may be performed by at least one of an electronic device (e.g., the electronic device 101 of FIG. 1, the electronic device 201 of FIGS. 2A, 2B, and 3) and at least one processor of the electronic device (e.g., the processor 120 of FIG. 1 or the processor 320 of FIG. 3). In an embodiment, at least one of operations 605 to 630 may be omitted, an order of some operations may be changed, another operation may be added, or at least two operations may be performed in parallel. FIGS. 7, 8, and 9A to 9C are referred to for better understanding of FIG. 6.

Referring to FIG. 6, in operation 605, the electronic device 201 may store user status information including user-related personal information, health information, and electronic medical records.

In an embodiment, the electronic device 201 may classify information included in status information of a user into personal information, health information, and electronic medical records to correspond to (or associate with) each of the first disclosure level to the third disclosure level as illustrated in FIG. 4, encrypt and store the same. The status information of a user may be stored in memory (e.g., the memory 330 of FIG. 3) of the electronic device 201 or a server (e.g., the server 208 of FIG. 2A) accessible by an external electronic device.

In an embodiment, the electronic device 201 may separately store user-related personal information, health information, and electronic medical records in memory (e.g., the memory 330 of FIG. 3) and/or a server (e.g., the server 208 of FIG. 2A) accessible by an external electronic device. In other words, the electronic device 201 may store information included in status information of a user without associating it with a disclosure level.

In operation 610, the electronic device 201 may monitor a driving state using information related to driving of a vehicle. According to an embodiment, an operation of monitoring a driving state may be performed in a background state. The electronic device 201 provides an activity recognition function, and for example, may distinguish and provide a stationary state (e.g., a sedentary/stationary state), a cycling state (e.g., a cycling state), or a state of riding in a vehicle such as an automobile (e.g., an in vehicle state). A vehicle riding state (e.g., an in vehicle state) may be activated (or turned on) when the electronic device 201 is connected to the vehicle 210 by Bluetooth, and accordingly, a vehicle function (e.g., a speaker call function while driving) corresponding to a vehicle state may be provided.

According to an embodiment, in a vehicle riding state (e.g., an in vehicle state), a driving state may be monitored by collecting various information (e.g., acceleration, Bluetooth, GPS, or Wi-Fi information). For example, when connected to the vehicle 210 by Bluetooth, when GPS speed information is a designated speed (e.g., about 10 m/s) and a Wi-Fi scanning list (or Wi-Fi AP) changes significantly, or based on at least one of pattern analysis of acceleration, the electronic device 201 may identify that it corresponds to a vehicle riding state (e.g., an in vehicle state).

Accordingly, in a vehicle riding state, when a value sensed through the sensor module 310 (e.g., an acceleration sensor, a gyro sensor) is equal to or greater than a threshold, it may be identified that a vehicle accident occurs rather than a simple fall accident. In this case, in recognizing a vehicle accident occurrence circumstance, various methods described above may be used in addition to the sensor module 310.

As such, a driving state may be monitored in a state of riding in a vehicle operating in the background (e.g., an in vehicle state), and accordingly, occurrence of a vehicle accident may also be detected. An operation of detecting occurrence of a vehicle accident while monitoring a driving state of a vehicle may be performed substantially identically in the wearable electronic device 202 as well as the electronic device 201.

Meanwhile, in the foregoing, a case of monitoring a driving state of a vehicle in a state of riding in a vehicle operating in the background (e.g., an in vehicle state) has been described as an example, but a driving state of a vehicle may also be monitored while a navigation application is being executed.

In operation 615, while monitoring a driving state, the electronic device 201 may identify whether a vehicle accident occurs.

In operation 620, in response to identifying occurrence of a vehicle accident, the electronic device 201 may notify occurrence of an accident. On the other hand, when a vehicle accident has not occurred, unless vehicle operation is terminated, an operation of monitoring a driving state may be periodically or aperiodically performed in operation 610.

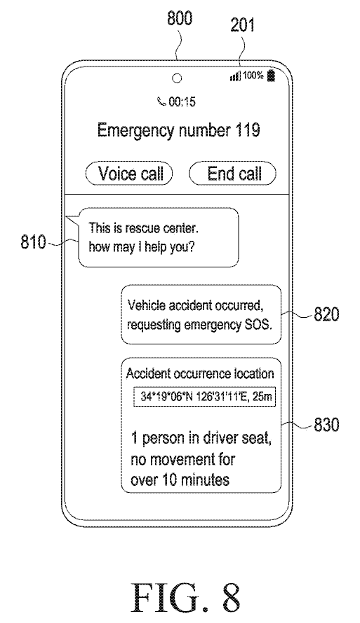

In an embodiment, the electronic device 201 may display a screen notifying occurrence of an accident as illustrated in FIGS. 7 or 8.

FIG. 7 is a view illustrating an example of a second screen upon occurrence of a vehicle accident according to an embodiment of the disclosure.

FIG. 8 is a view illustrating an example of a screen upon occurrence of a vehicle accident according to an embodiment of the disclosure.

Referring to FIG. 7, when occurrence of a vehicle accident is detected, the electronic device 201 may output a notification notifying occurrence of an accident as illustrated in 700a. A rescue notification may be selectively provided according to user feedback on the notification. For example, in the case of a minor accident, a user may select cancellation of an emergency contact in response to the notification. On the other hand, e.g., when a user does not select a 'cancel' button for a designated time (e.g., 10 seconds), it may be determined that a user is in a high-risk state where emergency rescue may not be requested, and as illustrated in 700b and 700c, an emergency call may be performed to a designated institution or a designated contact (e.g., family, 119).

Meanwhile, when emergency rescue may not be directly requested, even when an emergency call is performed, a user may be in a state in which a user may not have a conversation for rescue due to loss of consciousness. Therefore, as one of methods for delivering accident content to a rescue center during an emergency call, a function of answering a call as text may be used. For example, when occurrence of a vehicle accident is detected by the electronic device 201, a call may be automatically connected to a rescue center (e.g., 119), and since a user is unconscious, an operation of informing an emergency circumstance and requesting emergency rescue may be performed based on a learning model AI. For example, when a user has set (or activated) answering a call as text in a call setting, the learning model AI may inform that it is speaking on behalf of a user. FIG. 8 illustrates a screen 800 that is guided according to connection between the electronic device 201 and a rescue center.

Referring to FIG. 8, when a call is connected to a rescue center (e.g., 119) (810), the learning model AI may output content requesting rescue due to occurrence of a vehicle accident as voice to the rescue center (e.g., 119). Here, rescue request content may be displayed as text on the electronic device 201. Further, the learning model AI may output content related to accident occurrence as voice to the rescue center (e.g., 119). For example, content 820 notifying that a vehicle accident occurs and content 830 related to an accident occurrence location or a state of an accident victim may be output as voice to the rescue center (e.g., 119).

In an embodiment, an operation of converting text summarizing accident-related content into a designated voice or a voice learned with a user's voice using text-to-speech (TTS) technology and outputting it in a voice form may be performed in the learning model AI, but may also be performed in a generative AI model such as a large language model (LLM). FIG. 8 illustrates accident content informing an accident occurrence location and a state of an accident victim, but various information such as an accident type, accident intensity, or the number of passengers may be provided in a voice or text form based on sensed information in the electronic device 201 and/or the wearable electronic device 202.

In operation 625, the electronic device 201 may display vehicle accident information.

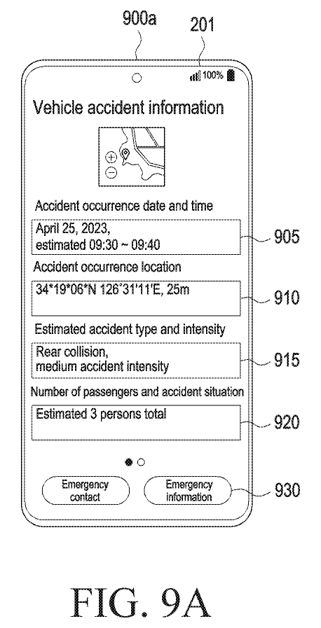

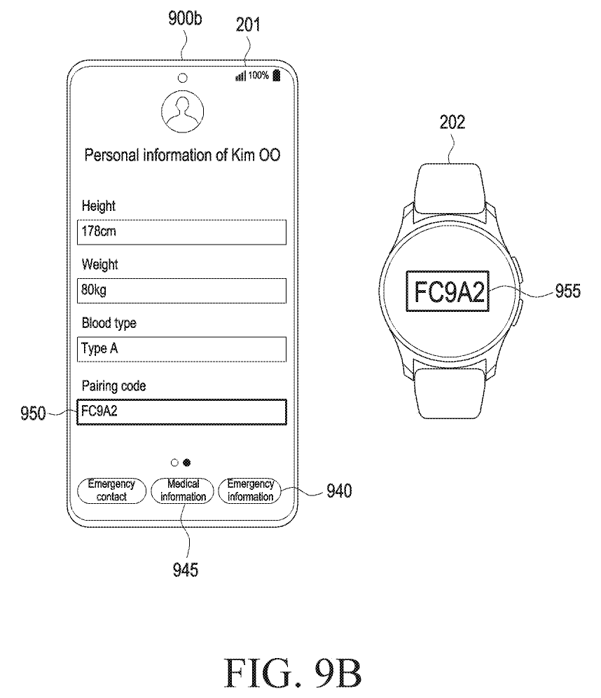

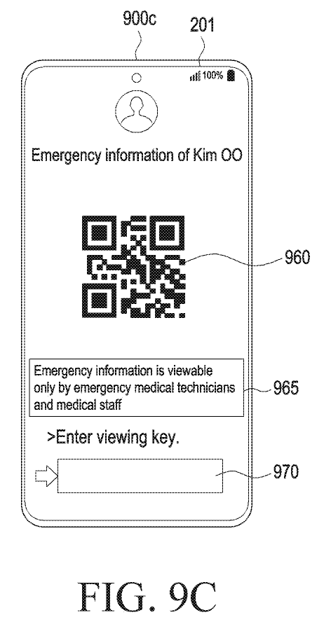

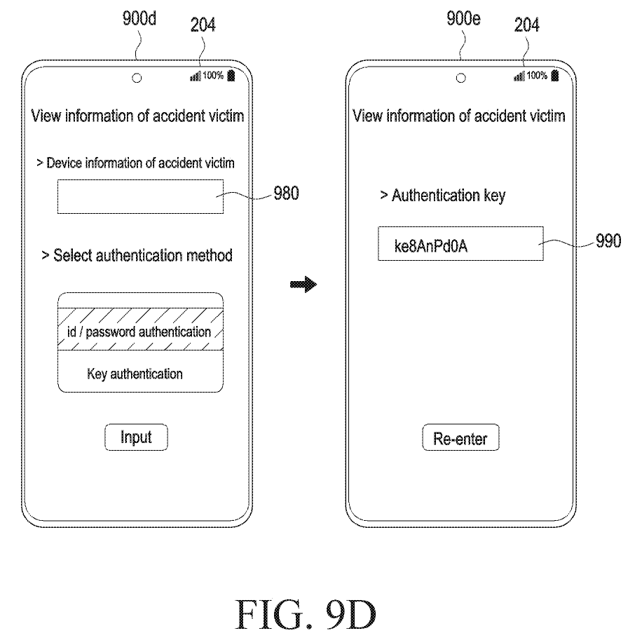

In an embodiment, the electronic device 201 may display a screen illustrating vehicle accident information as illustrated in FIG. 9A.

FIG. 9A is a view illustrating an example of a screen illustrating vehicle accident information according to an embodiment of the disclosure.

Referring to FIG. 9A, as illustrated in 900a, a notification illustrating vehicle accident information displayed by the electronic device 201 may include items illustrating an accident occurrence date and time 905 (or estimated accident time), an accident occurrence location 910 (or accident occurrence place), an accident type (e.g., front collision, rear collision, side collision, or rollover of a vehicle) and estimated accident intensity 915 (e.g., high, medium, low), the number of passengers, and an accident circumstance 920. Such vehicle accident information may be used by an emergency medical technician to grasp an accident circumstance after an emergency call.

In an embodiment, the electronic device 201 may provide an item 930 for displaying basic personal information of a user so that an emergency medical technician may identify a user's identity while displaying vehicle accident information. For example, when an emergency medical technician selects the item 930, the electronic device 201 may output a screen 900b illustrating personal information of a user as illustrated in FIG. 9B. For example, in response to detecting occurrence of a vehicle accident, the electronic device 201 may identify personal information of a user corresponding to a first disclosure level among status information of a user, and may provide the identified personal information together with vehicle accident information.

If there is a passenger in the vehicle in addition to a user, a process of matching whose device the electronic device 201 belongs to may be necessary. Accordingly, as illustrated in FIG. 9B, the electronic device 201 may display common information 950, 955 on each of the electronic device 201 and the wearable electronic device 202.

FIG. 9B is a view illustrating a wearable electronic device in a pairing state with an electronic device upon a vehicle accident according to an embodiment of the disclosure.

Meanwhile, in the foregoing, a case where personal information of a user is displayed as illustrated in FIG. 9B when an item 930 illustrating emergency information in FIG. 9A is selected has been illustrated, but a method of displaying personal information of a user may not be limited thereto. For example, the electronic device 201 may display a screen 900a illustrating vehicle accident information so that personal information of a user is also not exposed, and upon selection of an item 930 illustrating emergency information, may display a screen 900c for allowing only access to user status information by an authenticated target as illustrated in FIG. 9C.

FIG. 9C is a view illustrating an example of a screen illustrating an indicator for providing vehicle accident-related user status information according to an embodiment of the disclosure.

In an embodiment, the electronic device 201 may provide an item 945 illustrating medical information and an item 940 illustrating emergency information on a screen 900b on which personal information of a user is provided. Accordingly, upon selection of the item 945 illustrating medical information or the item 940 illustrating emergency information, the electronic device 201 may display a screen 900c for allowing only access to user status information by an authenticated target as illustrated in FIG. 9C.

In operation 630, the electronic device 201 may display an indicator for providing status information of a user so as to be accessible by an authenticated external user.

In an embodiment, the electronic device 201 may display a screen 900c including an indicator 960 for providing status information of a user and a message 965 informing that viewing by an emergency medical technician or a medical staff is possible as illustrated in FIG. 9C. Further, an input window 970 for a viewing key for an emergency medical technician or a medical staff to view status information of a user may be displayed. Here, the viewing key may be an authentication key issued to an emergency medical technician or a medical staff, and may also be referred to as an accessor key for accessing status information of a user. Such a viewing key may be issued through authentication with a server (e.g., the server 208 of FIG. 2A) managing status information of a user to an emergency medical technician or a medical staff upon occurrence of an accident, and may be information (or a key) shared in advance through separate authentication. For example, pre-shared authentication information (e.g., a medical staff number, an ambulance operation notification (report) number) may be input into the viewing key input window 970.

The viewing key may be encrypted and displayed so as not to be exposed to an external user. Upon a viewing request using the indicator 960, the viewing key may be delivered to an emergency medical technician or a medical staff through a specific application. Further, when an emergency medical technician or a medical staff is in a state of receiving a viewing key through authentication, the emergency medical technician or medical staff may directly input a viewing key (or authentication key) through a window 970 of the electronic device 201 to view status information of a user.

In an embodiment, the electronic device 201 may perform authentication on a target to disclose status information of a user, i.e., an emergency medical technician or a medical staff, using the input viewing key. Further, the viewing key input through the electronic device 201 may be delivered to the server 208, and the server 208 may authenticate the input viewing key and provide the authentication result to the electronic device 201.

In an embodiment, a viewing key required for viewing status information of a user may be issued in advance by an emergency medical technician or a medical staff through authentication with the server 208, and a method of issuing a viewing key (or authentication key) may not be limited thereto.

FIG. 9D is referred to illustrating an example of a method for an emergency medical technician or a medical staff to receive a viewing key through authentication.

FIG. 9D is a view illustrating an example of a screen displayed on an external electronic device of an emergency medical technician and/or a medical staff according to an embodiment of the disclosure.

Referring to FIG. 9D, e.g., when an emergency medical technician who has arrived at an accident scene captures (or scans) an indicator (e.g., the indicator 960 of FIG. 9C) for providing status information of a user using an external electronic device 204, i.e., his or her own device, as illustrated in 900d, a specific key may be automatically input into a device information input window 980 of an accident victim's device, i.e., the electronic device 201. Further, an authentication method selection window is a window for selecting a method for authenticating that a user of a specific application is an emergency medical technician or a medical staff, and an emergency medical technician or a medical staff may complete identity authentication. Thereafter, when an 'input' button is selected, the server 208 generates an authentication key for an emergency medical technician or a medical staff, and the external electronic device 204 may obtain the generated authentication key from the server 208 and display the authentication key 990 on an application screen as illustrated in 900e.

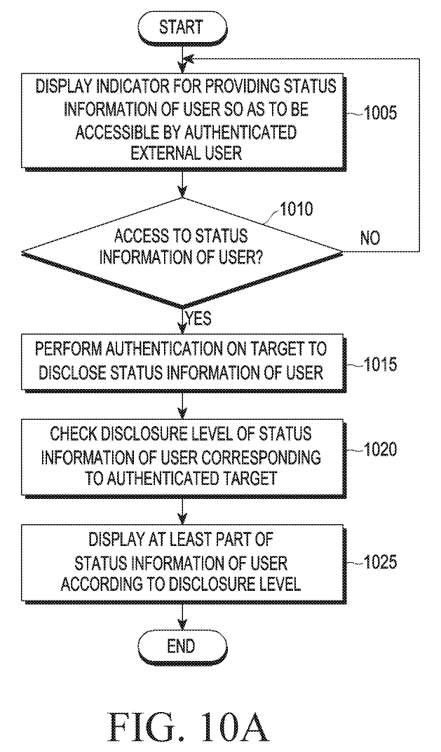

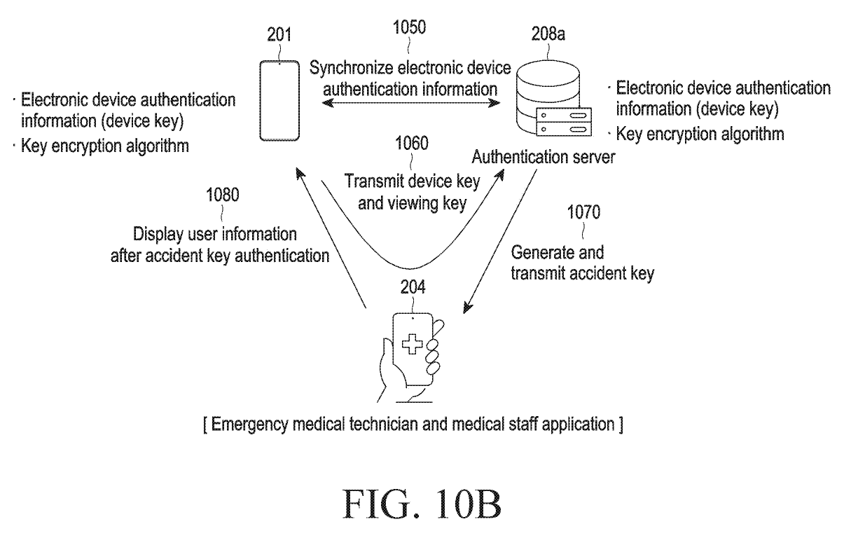

In an embodiment, after authentication as an emergency medical technician by inputting the authentication key 990 generated as described above into the input window 970 of FIG. 9C, at least a part of user status information may be viewed at a disclosure level corresponding to an access authority of an emergency medical technician. In the case of a medical staff, in order to actually view status information of a user, after authentication as a medical staff through the indicator, at least a part of user status information may be viewed at a disclosure level corresponding to an access authority of a medical staff. FIGS. 10A and 10B are referred to in order to examine in detail a method of authenticating an external electronic device of an external user such as an emergency medical technician or a medical staff.

FIG. 10A is an operation flowchart of an electronic device for providing vehicle accident-related user status information with different disclosure levels according to an embodiment of the disclosure.

FIG. 10B is a view illustrating an authentication method according to an embodiment of the disclosure.

Referring to FIG. 10A, an operation method may include operations 1005 to 1025. Each operation of the operation method of FIG. 10A may be performed by at least one of an electronic device (e.g., the electronic device 101 of FIG. 1, the electronic device 201 of FIGS. 2A and 3) and at least one processor of the electronic device (e.g., the processor 120 of FIG. 1 or the processor 320 of FIG. 3). In an embodiment, at least one of operations 1005 to 1025 may be omitted, an order of some operations may be changed, another operation may be added, or at least two operations may be performed in parallel. FIGS. 10B, and 11 to 13 are referred to for better understanding of FIG. 10A.

Referring to FIG. 10A, in operation 1005, the electronic device 201 may display an indicator (e.g., the indicator 960 of FIG. 9C) for providing status information of a user so as to be accessible by an authenticated external user.

In operation 1010, the electronic device 201 may identify whether there is an access to status information of a user. The access may be made by an external electronic device possessed by an emergency medical technician or a medical staff.

In operation 1015, in response to an access to status information of a user, the electronic device 201 may perform authentication on a target to disclose status information of a user.

Referring to FIG. 10B, the electronic device 201 may periodically perform an operation of synchronizing authentication information in advance with a server 208a (e.g., the server 208 of FIG. 2A) managing status information of a user. Here, each of the electronic device 201 and the server 208a may encrypt or decrypt authentication information using an encryption and decryption algorithm, thereby enhancing security of authentication information. For example, the electronic device 201 may transmit and synchronize (1050) authentication information such as a QR code to the server 208. Here, authentication information such as a QR code may be periodically generated by the electronic device 201. If a communication connection between the electronic device 201 and the server 208a is released or a vehicle accident occurs, the most recently generated authentication information in the electronic device 201 may be used to perform authentication on a target to disclose status information of a user. The most recently generated authentication information in the electronic device 201 may be referred to as a device key. Such a device key may also be possessed by the server 208a through synchronization, and may be used to restrict viewing so that an emergency medical technician or a medical staff may view status information of a user only when an actual accident occurs.

If occurrence of a vehicle accident is detected, the electronic device 201 may display an indicator 960 for providing status information of a user and a window 970 for inputting a viewing key so as to be accessible only by an emergency medical technician or a medical staff as illustrated in 900c of FIG. 9C. An emergency medical technician or a medical staff may input a viewing key (or accessor key) (e.g., an identifier (ID) and password, an authentication key). Further, the viewing key may include pre-shared authentication information (e.g., a medical staff number, an ambulance operation notification (report) number). A device key (or QR code) and a viewing key may be transmitted (1060) to the server 208a, and the server 208a may encrypt the device key (or QR code) and viewing key provided through a specific application and generate an authentication key (or accident key) required for viewing status information of a user and transmit (1070) it to the external electronic device 204 of an emergency medical technician or a medical staff.

When an emergency medical technician or a medical staff inputs the provided authentication key (or accident key) to the electronic device 201, the electronic device 201 may identify whether information decrypted from the authentication key (or accident key) through a decryption algorithm matches authentication information (or device key) possessed by the electronic device 201. If the decrypted information matches authentication information (or device key) possessed by the electronic device 201, by checking whether a target wishing to view status information of a user is an emergency medical technician or a medical staff and providing an authentication key corresponding to the target, an emergency medical technician or a medical staff may be able to view (1080) step-by-step information on a user status. As such, an operation of performing authentication on a target to disclose status information of a user may use a key generated by the server 208a, but an authentication method thereof may not be limited thereto.

On the other hand, when there is no access to status information of a user, in operation 1005, the electronic device 201 may maintain display of the indicator. Further, when there is no access to status information of a user until a predetermined time elapses, an operation of displaying an indicator for providing status information of a user may be terminated.

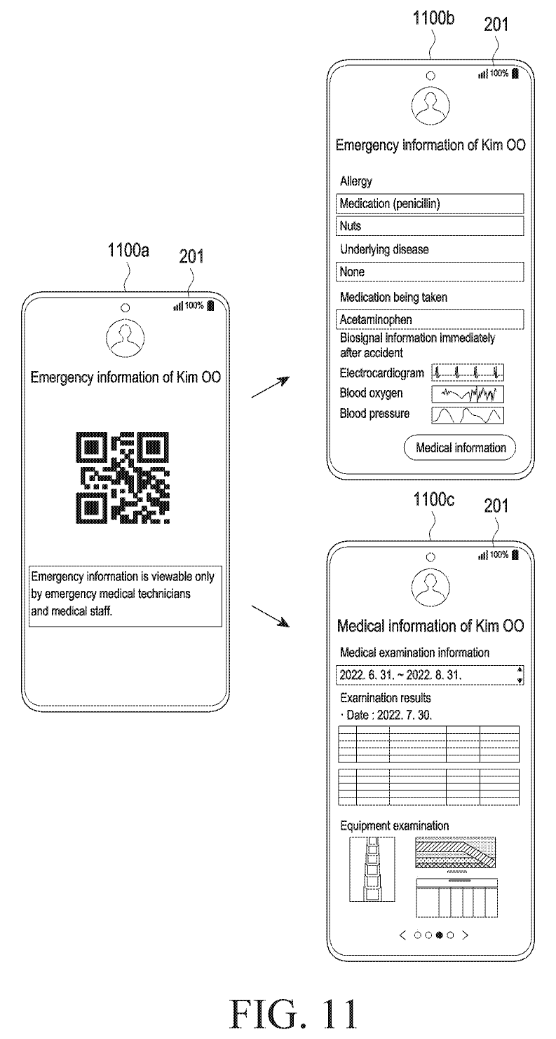

In an embodiment, an access by an external electronic device may indicate an operation in which an external electronic device scans (or captures) an indicator being displayed on the electronic device 201. For example, the electronic device 201 may display an indicator for providing status information of a user as illustrated in 1100a of FIG. 11.

FIG. 11 is a view illustrating an example of a screen on which vehicle accident-related user status information with different disclosure levels is provided for each authenticated target using one indicator according to an embodiment of the disclosure.

According to the scan operation, a screen for inputting authentication information for authenticating whether a target capable of viewing may be displayed on an external electronic device. Accordingly, an emergency medical technician or a medical staff may input pre-shared authentication information (e.g., a medical staff number, an ambulance operation notification (report) number).

The authentication information input from the external electronic device may be transmitted to a server (e.g., the server 208 of FIG. 2A) managing status information of a user, and authentication information may be provided to the electronic device 201 through the server. For example, the electronic device 201 may perform authentication on a target to disclose status information of a user based on authentication information provided through the server.

In an embodiment, instead of the electronic device 201, the server may perform authentication on an external electronic device using the authentication information. After the authentication is completed, the server may transmit a notification according to authentication completion to the electronic device 201, so that the electronic device 201 may also recognize authentication completion for a target to disclose status information of a user.

Meanwhile, in the foregoing, a QR code has been illustrated as an example of an indicator for providing status information of a user as illustrated in 1100a, but in order to prevent exposure of sensitive information, an indicator with status information of a user security-processed, e.g., blinded with '*', may also be displayed. Accordingly, an emergency medical technician or a medical staff may know that provision of status information of a user is possible through a screen on which security-processed status information of a user is displayed, but may not be able to actually check status information of a user. In such a case, by inputting authentication information (e.g., an authentication key) on a screen (e.g., the viewing key input window 970 as illustrated in FIG. 9C) of the electronic device 201 by an emergency medical technician or a medical staff, the electronic device 201 may perform authentication on a target to disclose status information of a user using the input authentication information. For example, an emergency medical technician and a medical staff may each have different authentication information.

In operation 1020, the electronic device 201 may check a disclosure level of status information of a user corresponding to an authenticated target. For example, the electronic device 201 may identify whether it is an external electronic device of an emergency medical technician or an external electronic device of a medical staff using the input authentication information. Accordingly, the electronic device 201 may determine a disclosure level corresponding to an identified external electronic device.

In an embodiment, when information included in status information of a user is stored without being associated with a disclosure level, in response to an access by an external electronic device upon occurrence of a vehicle accident, the electronic device 201 may identify an access authority of the external electronic device and determine a disclosure level corresponding to the access authority, thereby identifying (or extracting, generating) information corresponding to the disclosure level among status information of a user.

For example, the learning model AI may infer, based on vehicle accident information and status information of a user, whether an emergency circumstance of a user is a circumstance requiring emergency treatment by an emergency medical technician or a circumstance requiring diagnosis and treatment by hospital medical staff. The learning model AI may check information necessary in an inferred circumstance among status information of a user and may classify checked information according to disclosure levels.

In operation 1025, the electronic device 201 may display at least a part of status information of a user according to a disclosure level. For example, in response to identifying a second disclosure level according to an access authority of an external electronic device of an emergency medical technician, the electronic device 201 may display health information recorded by a user necessary for emergency treatment among status information of a user as illustrated in 1100b. In response to identifying a third disclosure level according to an access authority of an external electronic device of a medical staff, the electronic device 201 may display electronic medical records necessary for diagnosis and treatment among status information of a user as illustrated in 1100c.

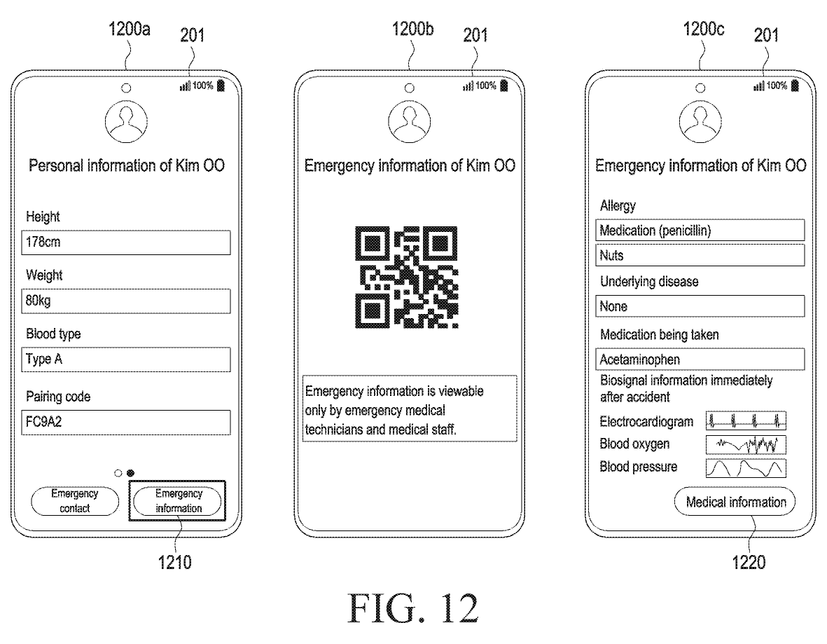

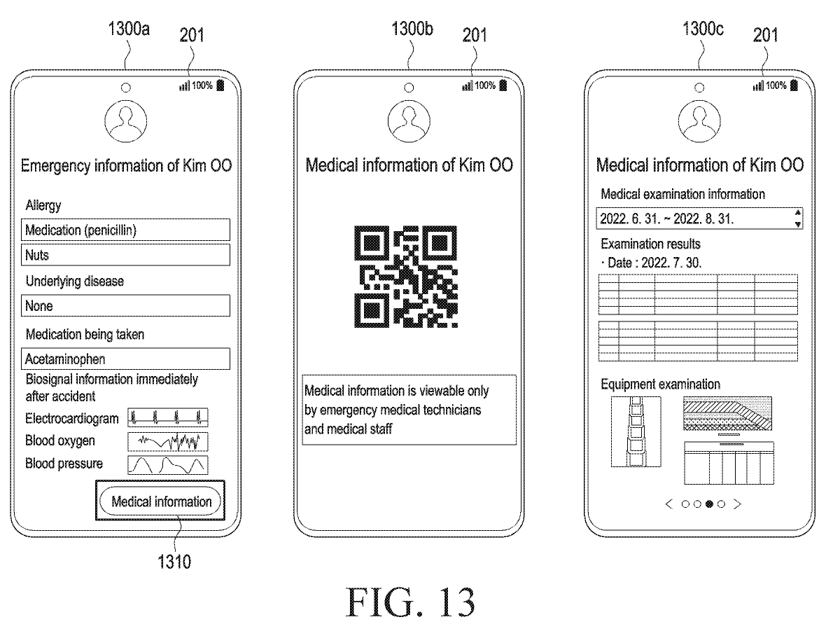

Meanwhile, in 1100a of FIG. 11, a case where when authentication is completed using one indicator, at least a part of user status information is differently provided at different disclosure levels for each authenticated target as illustrated in 1100b and 1100c, screens entering for displaying user status information for each disclosure level may be differently displayed as illustrated in FIGS. 12 and 13.

FIG. 12 is a view illustrating an example of a screen on which health information among user status information is provided according to an embodiment of the disclosure.

FIG. 13 is a view illustrating an example of a screen on which electronic medical records among user status information are provided according to an embodiment of the disclosure.

Referring to FIG. 12, upon a vehicle accident, the electronic device 201 may provide an item 1210 illustrating emergency information on a screen 1200a displaying personal information of a user. If the item 1210 is selected by an external user, the electronic device 201 may display a screen 1200b for providing emergency information (e.g., health information recorded by a user). Here, even when the item 1210 illustrating emergency information is selected, since it is not known whether a target requesting emergency information is an authenticated target, the electronic device 201 may display an indicator indicating security-processed status of a user. The electronic device 201 may authenticate a target that has accessed the emergency information using the indicator. If it is identified that a target that has accessed the emergency information is an external electronic device of an emergency medical technician, the electronic device 201 may display a screen 1200c including emergency information about a user. Here, emergency information includes information necessary for emergency treatment by an emergency medical technician, and in the case of a medical staff, information at a higher disclosure level may be required. For this purpose, an item 1220 illustrating medical information may be provided.

For example, in response to selection of an item 1310 illustrating medical information as illustrated in 1300a of FIG. 13, the electronic device 201 may display a screen 1300b for providing medical information (e.g., electronic medical records). The electronic device 201 may authenticate a target that has accessed the medical information using the indicator. If it is identified that a target that has accessed the medical information is an external electronic device of a medical staff, the electronic device 201 may display a screen 1300c including medical information about a user.

Meanwhile, in the foregoing, a case where screens with different disclosure levels are displayed according to a target that has accessed status information of a user has been described as an example, but screens with different disclosure levels may also be displayed according to a time of emergency transport or a time of arrival at a medical institution using sensing information by the sensor module 310 and location information (e.g., GPS information) of an electronic device.

For example, the electronic device 201 may check at least one or a combination of whether a target that has accessed emergency information is an external electronic device of an emergency medical technician or a time of emergency transport. When a time of emergency transport is identified, the electronic device 201 may provide health information of a user necessary for emergency treatment in response to an access by an external electronic device of an emergency medical technician. Further, the electronic device 201 may provide health information of a user necessary for emergency treatment in response to identifying that it is an access by an external electronic device of an emergency medical technician and identifying a time of emergency transport. Further, based on identifying that a target that has accessed medical information is an external electronic device of a medical staff, identifying a time of arrival at a medical institution, or a combination thereof, the electronic device 201 may provide electronic medical records of a user necessary for diagnosis and treatment in response to an access by an external electronic device of a medical staff.

According to an embodiment, upon occurrence of a vehicle accident, information about a user of an electronic device may be minimized from being exposed to an unauthorized external user, thereby preventing privacy infringement.

The electronic device according to various embodiments of the disclosure may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. As used herein, each of such phrases as "A or B," "at least one of A and B," “at least one of A or B,” "A, B, or C," "at least one of A, B, and C," and “at least one of A, B, or C,” may include all possible combinations of the items enumerated together in a corresponding one of the phrases. As used herein, such terms as "1st" and "2nd," or “first” and "second" may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” "coupled to," "connected with," or “connected to” another element (e.g., a second element), it means that the element may be coupled with the other element directly (e.g., wiredly), wirelessly, or via a third element.

As used herein, the term "module" may include a unit implemented in hardware, software, or firmware, and may interchangeably be used with other terms, for example, "logic," "logic block," "part," or "circuitry". A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a compiler or a code executable by an interpreter. The storage medium readable by the machine may be provided in the form of a non-transitory storage medium. Wherein, the term "non-transitory" simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store^{TM}), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. Some of the plurality of entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

According to an embodiment, in a storage medium storing at least one computer-readable instruction, the at least one instruction, when executed by at least one processor 120, 320 of an electronic device 101, 201, causes the electronic device to perform at least one operation, and the at least one operation may include an operation of identifying that a user is in a vehicle 210.

According to an embodiment, the at least one operation may include an operation of displaying an indicator for providing status information of a user, based on an occurrence of a vehicle accident being detected while monitoring a driving state of the vehicle using at least one sensor of the electronic device.

According to an embodiment, the at least one operation may include an operation of providing at least a part of status information of a user in response to an access by an external electronic device to status information of a user using the indicator.

According to an embodiment, the operation of providing at least a part of status information of a user may include an operation of determining a disclosure level of status information of a user in response to an access by an external electronic device to status information of a user using the indicator, and an operation of displaying at least a part of status information of a user based on the determined disclosure level.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device individually or collectively, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a display;
at least one sensor;
at least one processor operatively connected to the display and the at least one sensor; and
memory storing instructions that, when executed by the at least one processor individually or collectively, cause the electronic device to:
display an indicator for providing status information of a user on the display, based on an occurrence of a vehicle accident being detected while monitoring a driving state of a vehicle using the at least one sensor, and
in response to an access to the status information of the user in the vehicle by an external electronic device using the indicator, provide at least a part of the status information of the user.

2. The electronic device of claim 1, wherein the instructions, when executed by the at least one processor individually or collectively, further cause the electronic device to:
in response to the access by the external electronic device to the status information of the user using the indicator, identify a disclosure level of the status information of the user; and
display at least a portion of the status information of the user on the display, based on the identified disclosure level.

3. The electronic device of claim 2, wherein the status information of the user includes personal information of the user, health information recorded by the user, or electronic medical records of the user, and is stored in the memory or on a server accessible by the external electronic device.

4. The electronic device of claim 3, wherein the instructions, when executed by the at least one processor individually or collectively, further cause the electronic device to:
identify the disclosure level of the status information of the user according to an access authority of the external electronic device;
based on the identified disclosure level being a first disclosure level, display the personal information of the user among the status information of the user on the display;
based on the identified disclosure level being a second disclosure level, display the health information recorded by the user among the status information of the user on the display; and
based on the identified disclosure level being a third disclosure level, display the electronic medical records of the user among the status information of the user on the display.

5. The electronic device of claim 4, wherein the instructions, when executed by the at least one processor individually or collectively, further cause the electronic device to:
in response to the access by the external electronic device to the status information of the user using the indicator, identify a time of emergency transport or a time of arrival at a medical institution, based on sensing information by the at least one sensor and location information of the electronic device;
based on identifying the time of emergency transport, display the health information recorded by the user among the status information of the user on the display; and
based on identifying the time of arrival at the medical institution, display the electronic medical records of the user among the status information of the user on the display.

6. The electronic device of claim 1, wherein the indicator for providing the status information of the user includes a unique encrypted quick response (QR) code to be used to access the status information of the user.

7. The electronic device of claim 1, further comprising:
communication circuitry,
wherein the instructions, when executed by the at least one processor individually or collectively, further cause the electronic device to:
in case that the electronic device is connected to a wearable electronic device worn by the user through the communication circuitry, identify an abnormal condition of the user based on a biosignal of the user provided from the wearable electronic device, based on detecting the occurrence of the vehicle accident, and
based on the identified abnormal condition of the user, perform an emergency call.

8. The electronic device of claim 7, wherein the instructions, when executed by the at least one processor individually or collectively, further cause the electronic device to:
in case that the electronic device is connected to the wearable electronic device worn by the user through the communication circuitry, display a specific code, which is common to the wearable electronic device, on the display to indicate a pairing state between the electronic device and the wearable electronic device.

9. The electronic device of claim 8, wherein the instructions, when executed by the at least one processor individually or collectively, further cause the electronic device to:
identify whether the user is in the vehicle using at least one of the at least one sensor or the communication circuitry; and
based on identifying that the user is in the vehicle, monitor the driving state of the vehicle.

10. A method for providing vehicle accident-related user status information in an electronic device, the method comprising:
displaying an indicator for providing status information of a user in a vehicle, based on an occurrence of a vehicle accident being detected while monitoring a driving state of the vehicle using at least one sensor of the electronic device; and
in response to an access to the status information of the user by an external electronic device using the indicator, providing at least a part of the status information of the user.

11. The method of claim 10, wherein the providing of the at least the part of the status information of the user includes:
in response to the access by the external electronic device to the status information of the user using the indicator, identifying a disclosure level of the status information of the user; and
based on the identified disclosure level, displaying at least a portion of the status information of the user.

12. The method of claim 11, wherein the status information of the user includes personal information of the user, health information recorded by the user, or electronic medical records of the user, and is stored in memory or on a server accessible by the external electronic device.

13. The method of claim 12, wherein the providing of the at least the part of the status information of the user includes:
identifying the disclosure level of the status information of the user according to an access authority of the external electronic device;
based on the identified disclosure level being a first disclosure level, displaying the personal information of the user among the status information of the user;
based on the identified disclosure level being a second disclosure level, displaying the health information recorded by the user among the status information of the user; and
based on the identified disclosure level being a third disclosure level, displaying the electronic medical records of the user among the status information of the user.

14. The method of claim 13, further comprising:
in response to the access by the external electronic device to the status information of the user using the indicator, identifying a time of emergency transport or a time of arrival at a medical institution, based on sensing information by the at least one sensor and location information of the electronic device;
based on identifying the time of emergency transport, displaying the health information recorded by the user among the status information of the user; and
based on identifying the time of arrival at the medical institution, displaying the electronic medical records of the user among the status information of the user.

15. The method of claim 10, wherein the indicator for providing the status information of the user includes a unique encrypted quick response (QR) code to be used to access the status information of the user.

16. The method of claim 10, further comprising:
in case that the electronic device is connected to a wearable electronic device worn by the user through a communication circuitry, identifying an abnormal condition of the user based on a biosignal of the user provided from the wearable electronic device, based on detecting the occurrence of the vehicle accident, and
based on the identified abnormal condition of the user, performing an emergency call.

17. The method of claim 16, further comprising:
in case that the electronic device is connected to the wearable electronic device worn by the user through the communication circuitry, displaying a specific code, which is common to the wearable electronic device, on the display to indicate a pairing state between the electronic device and the wearable electronic device.

18. The method of claim 17, further comprising:
identifying whether the user is in the vehicle using at least one of the at least one sensor or the communication circuitry; and
based on identifying that the user is in the vehicle, monitoring the driving state of the vehicle.

19. One or more non-transitory computer-readable storage media storing one or more computer programs including computer-executable instructions that, when executed by one or more processors of an electronic device individually or collectively, cause the electronic device to perform operations, the operations comprising:
displaying an indicator for providing status information of a user in a vehicle, based on an occurrence of a vehicle accident being detected while monitoring a driving state of the vehicle using at least one sensor of the electronic device; and
in response to an access to the status information of the user by an external electronic device using the indicator, providing at least a part of the status information of the user.

20. The one or more non-transitory computer-readable storage media of claim 19, the operations further comprising:
in response to the access by the external electronic device to the status information of the user using the indicator, identifying a disclosure level of the status information of the user; and
based on the identified disclosure level, displaying at least a portion of the status information of the user.
